(19) United States
(12) Patent Application Publication
(10) Pub. No.: US 2010/0282264 A1
(43) Pub. Date: Nov. 11, 2010

(54) RESPIRATORY MASK HAVING A DAMPING RING

(75) Inventor: Eric Chang, Taichung Hsien (TW)

Correspondence Address: PEACOCK MYERS, P.C., 201 THIRD STREET, N.W., SUITE 1340, ALBUQUERQUE, NM, 87102, US

(73) Assignee: Hsiner Company, Ltd., Taichung Hsien (TW)

(21) Appl. No.: 12/435,910

(22) Filed: May 5, 2009

(51) Int. Cl. A61M 16/06

(52) U.S. Cl. 128/206.21

(57) ABSTRACT

A respiratory mask includes a mask body that is adapted to be attached to a patient's face and that includes a connecting hole, a vent tube that has one end extending into the connecting hole, a flexible damping ring that is disposed in the connecting hole and that has an inner periphery around the vent tube, and an outer periphery surrounding the inner periphery, a first fixing unit that fixes the inner periphery of the damping ring to the vent tube, and a second fixing unit that fixes the outer periphery of the damping ring to the mask body.

### BACKGROUND OF THE INVENTION

1. Field of the Invention

This invention relates to a respiratory mask, more particularly to a respiratory mask that includes a damping ring.

2. Description of the Related Art

Generally, a medical treatment for a patient suffering from sleep apnea includes using a respiratory mask to cover the patient's nose, or both of the patient's nose and mouth, and providing a positive airway pressure, thereby continuously enabling the patient to breathe smoothly during sleep.

Referring to FIG. 1, a conventional respiratory mask 1 includes a vent tube 11, a mask body 12 that is adapted to be attached to a patient's face and that is connected to the vent tube 11, two top positioning members 13 that are symmetrically spaced apart and that are fixedly connected to the mask body 12, two bottom positioning members 14 that are respectively disposed below the top positioning members 13, and a plurality of straps 15 that are respectively connected to the top and bottom positioning members 13,14, and that are adapted to wrap around the patient's head.

By virtue of the top and bottom positioning members 13,14, and the straps 15, the mask body 12 is capable of securely covering the patient's nose, or both of the patient's nose and mouth. A positive airway pressure can be further applied to the patient through the vent tube 11 and the mask body 12. Therefore, the patient wearing the respiratory mask 1 is continuously able to breathe smoothly during sleep.

However, when the patient has head movement or adjusts a sleeping position thereof during sleep, the mask body 12 attached to the patient's face may be displaced from its original position. The patient may feel uncomfortable since a force (e.g., a frictional force) is exerted on the patient's face when the mask body 12 is displaced relative to the patient's face. Furthermore, when the patient's limb accidentally moves the vent tube 11, the mask body 12 connected to the vent tube 11 may also be displaced relative to the patient's face due to the movement of the vent tube 11, thereby resulting in discomfort and inconvenience.

### SUMMARY OF THE INVENTION

The object of the present invention is to provide a respiratory mask that can overcome the aforesaid drawbacks of the prior art.

According to this invention, a respiratory mask includes a mask body that is adapted to be attached to a patient's face and that includes a connecting hole, a vent tube that has one end extending into the connecting hole, a flexible damping ring that is disposed in the connecting hole and that has an inner periphery around the vent tube, and an outer periphery surrounding the inner periphery, a first fixing unit that fixes the inner periphery of the damping ring to the vent tube, and a second fixing unit that fixes the outer periphery of the damping ring to the mask body.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a schematic diagram of a conventional respiratory mask in a state of use;

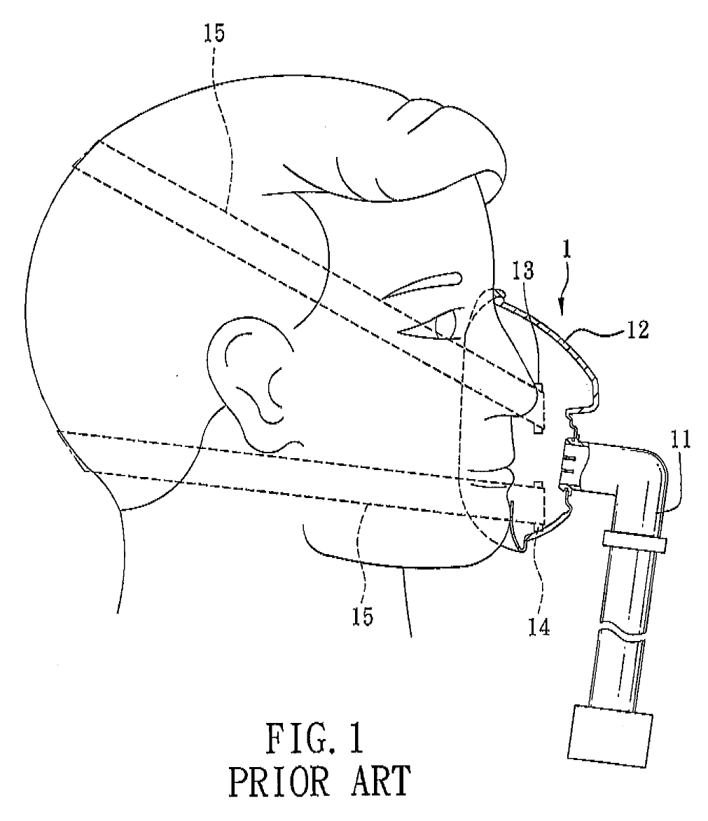

FIG. 2 is a fragmentary sectional view of the preferred embodiment of a respiratory mask according to this invention;

FIG. 3 is a fragmentary exploded sectional view of the preferred embodiment;

FIG. 4 is an enlarged fragmentary sectional view of the preferred embodiment; and

FIG. 5 is the same view as FIG. 2 but illustrating that a damping ring of the respiratory mask compresses at a left side and expands at a right side due to movement of a vent tube of the respiratory mask.

### DETAILED DESCRIPTION OF THE PREFERRED EMBODIMENT

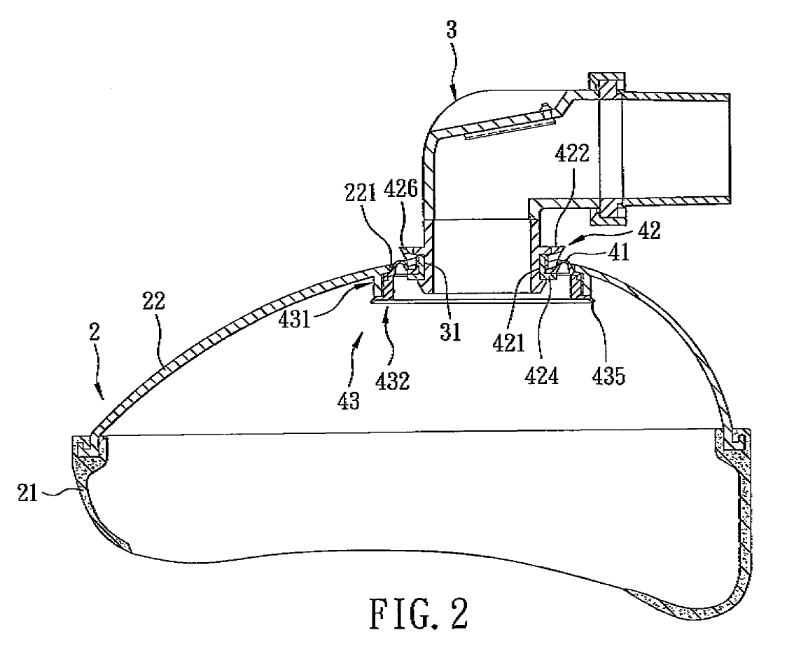

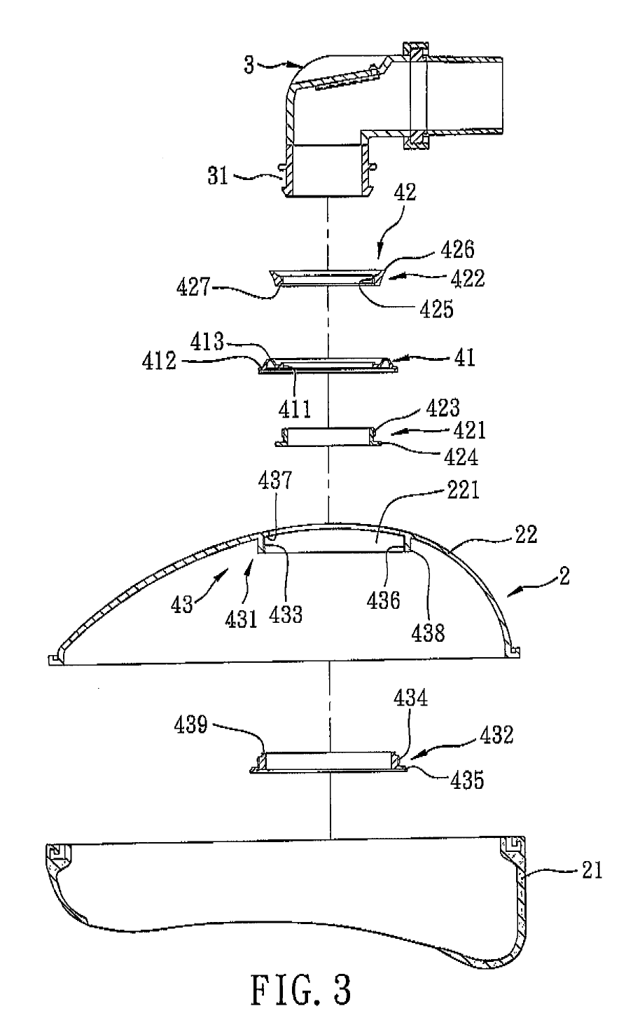

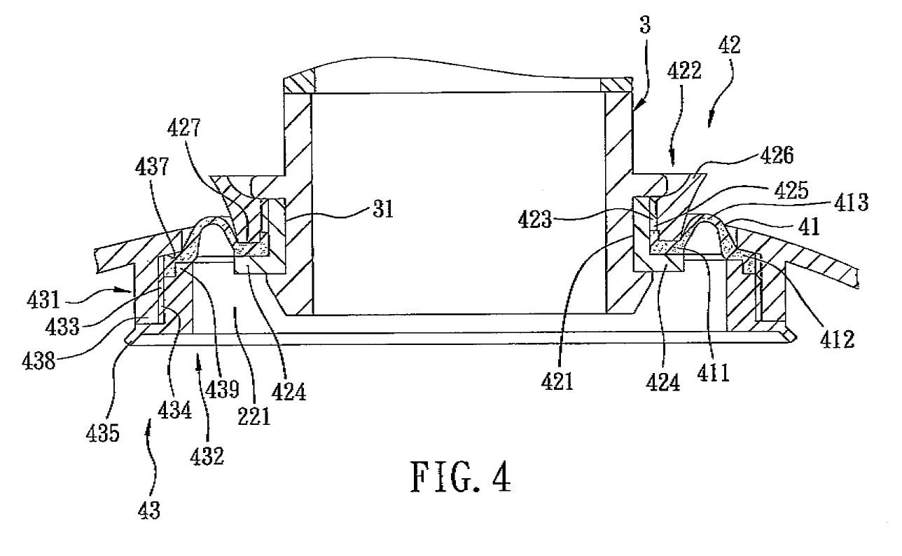

Referring to FIGS. 2, 3, and 4, the preferred embodiment of a respiratory mask according to the present invention includes a mask body 2, a vent tube 3, a flexible damping ring 41, a first fixing unit 42, and a second fixing unit 43.

The mask body 2 is adapted to be attached to a patient's face and includes a connecting hole 221. In this embodiment, the mask body 2 has a face contact portion 21, and a tube connection portion 22 that is connected to the face contact portion 21 and that has the connecting hole 221.

The vent tube 3 has one end that extends into the connecting hole 221. The one end of the vent tube 3 has two annular protrusions that project radially and outwardly from an outer surface of the vent tube 3, and that are spaced apart axially to form an annular groove 31 therebetween.

The damping ring 41 is made of silicone rubber, is disposed in the connecting hole 221, and has an inner periphery 411 around the vent tube 3, and an outer periphery 412 surrounding the inner periphery 411. In this embodiment, the damping ring 41 further has a folded flexible intermediate web 413 that interconnects the inner and outer peripheries 411,412. It should be noted that the damping ring 41 could be made of other materials, and that the intermediate web 413 could have other shapes.

The first fixing unit 42 fixes the inner periphery 411 of the damping ring 41 to the vent tube 3, and includes a first fixing inner ring 421 and a first fixing outer ring 422. The first fixing inner ring 421 has an outer surface that is formed with an external screw thread 423, and is disposed around an outer periphery of the vent tube 3. More specifically, the first fixing inner ring 421 is received fittingly between the annular protrusions of the end of the vent tube 3 (i.e., in the annular groove 31). In this embodiment, the first fixing inner ring 421 further has an annular flange 424 that projects outwardly and radially from the outer surface of the first fixing inner ring 421.

The first fixing outer ring 422 is disposed around the first fixing inner ring 421, engages the first fixing inner ring 421, and has an inner surface that is formed with an internal screw thread 425 engaging threadedly the external screw thread 423 of the first fixing inner ring 421. In this embodiment, the first fixing outer ring 422 further has a first press end 427 that extends axially toward the annular flange 424 of the first fixing inner ring 421, and a first handling end 426 that is opposite to the first press end 427 and that is operable to rotate the first fixing outer ring 422.

The second fixing unit 43 fixes the outer periphery 412 of the damping ring 41 to the mask body 2, and includes a second fixing internal ring 432 and a second fixing external ring 431. The second fixing internal ring 432 is disposed around the first fixing unit 42 and has an outer surface that is formed with an external screw thread 434.

The second fixing external ring 431 is formed on the mask body 2 around the second fixing internal ring 432, engages the second fixing internal ring 432, and has an inner surface 436 that is formed with an internal screw thread 433 threadedly engaging the external screw thread 434 of the second fixing internal ring 432. More specifically, the second fixing external ring 431 is formed in the tube connection portion 22, and has the inner surface 436 thereof defining the connecting hole 221. In this embodiment, the second fixing external ring 431 further has an annular bearing face 437 that projects from the inner surface 436 thereof into the connecting hole 221 in proximity to the damping ring 41, and a distal end 438 that is opposite to the annular bearing face 437.

Preferably, the second fixing internal ring 432 further has a second press end 439 that extends axially toward the annular bearing face 437 of the second fixing external ring 431 so as to press the outer periphery 412 of the damping ring 41 against the annular bearing face 437, and a second handling end portion 435 that is opposite to the second press end 439, that projects outwardly and radially to extend over the distal end 438 of the second fixing external ring 431, and that is operable to rotate the second fixing internal ring 432.

Assembly of the respiratory mask is described as follows. The damping ring 41 is sleeved on the first fixing inner ring 421. When the first fixing outer ring 422 is disposed around and rotated downwardly relative to the first fixing inner ring 421 by operating the first handling end 426, the internal screw thread 425 of the first fixing outer ring 422 engages threadedly the external screw thread 423 of the first fixing inner ring 421. Consequently, the inner periphery 411 of the damping ring 41 is clamped between the first press end 427 of the first fixing outer ring 422 and the annular flange 424 of the first fixing inner ring 421. In this embodiment, the inner periphery 411 of the damping ring 41 extends between the outer surface of the first fixing inner ring 421 and the inner surface of the first fixing outer ring 422 in proximity to the annular flange 424 of the first fixing inner ring 421. Subsequently, the outer periphery 412 of the damping ring 41 is disposed in contact with the annular bearing face 437 of the second fixing external ring 431. The second fixing internal ring 432 is inserted into and rotated upwardly relative to the second fixing external ring 431 by operating the second handling end portion 435 so that the internal screw thread 433 of the second fixing external ring 431 threadedly engages the external screw thread 434 of the second fixing internal ring 432. Thus, the outer periphery 412 of the damping ring 41 is clamped between the second press end 439 of the second fixing internal ring 432 and the annular bearing face 437 of the second fixing external ring 431. Finally, the end of the vent tube 3 is passed through the first fixing outer ring 422, the damping ring 41, and the first fixing inner ring 421 so as to fit the first fixing inner ring 421 in the annular groove 31, thereby completing the assembly of the respiratory mask.

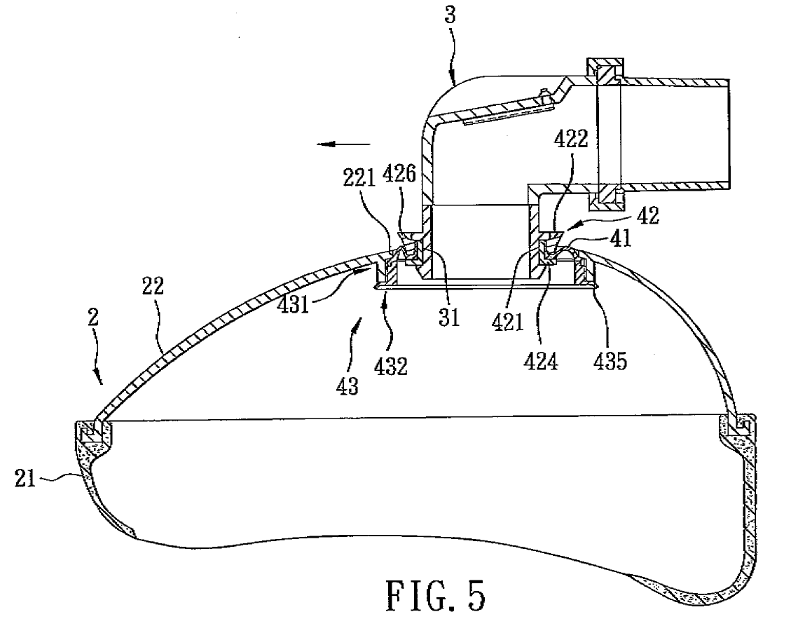

Referring to FIG. 5, when the respiratory mask worn by a sleeping patient is subjected a pulling or pushing force due to movement of the patient, the damping ring 41 between the vent tube 3 and the mask body 2 deforms (e.g., compresses at a left side and expands at a right side), thereby absorbing and dampening the pulling or pushing force, and reducing discomfort caused to the patient during sleep.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. A respiratory mask comprising:
a mask body adapted to be attached to a patient's face and including a connecting hole;
a vent tube having one end that extends into said connecting hole;
a flexible damping ring disposed in said connecting hole and having an inner periphery around said vent tube, and an outer periphery surrounding said inner periphery;
a first fixing unit fixing said inner periphery of said damping ring to said vent tube; and
a second fixing unit fixing said outer periphery of said damping ring to said mask body.

2. The respiratory mask as claimed in claim 1, wherein said first fixing unit includes a first fixing inner ring that is disposed around an outer periphery of said vent tube, and a first fixing outer ring that is disposed around said first fixing inner ring, and that engages said first fixing inner ring, said inner periphery of said damping ring being clamped between said first fixing inner and outer rings.

3. The respiratory mask as claimed in claim 2, wherein said first fixing inner ring has an outer surface that is formed with an external screw threads said first fixing outer ring having an inner surface that is formed with an internal screw thread engaging threadedly said external screw thread.

4. The respiratory mask as claimed in claim 3, wherein said first fixing inner ring further has an annular flange that projects outwardly and radially from said outer surface of said first fixing inner ring, said first fixing outer ring further having a first press end that extends axially toward said annular flange, said inner periphery of said damping ring being clamped between said first press end and said annular flange.

5. The respiratory mask as claimed in claim 4, wherein said inner periphery of said damping ring extends between said outer surface of said first fixing inner ring and said inner surface of said first fixing outer ring in proximity to said annular flange of said first fixing inner ring.

6. The respiratory mask as claimed in claim 5, wherein said first fixing outer ring further has a first handling end that is opposite to said first press end and that is operable to rotate said first fixing outer ring.

7. The respiratory mask as claimed in claim 2, wherein said one end of said vent tube has two annular protrusions that project radially and outwardly from an outer surface of said vent tube, and that are spaced apart axially, said first fixing inner ring being received fittingly between said annular protrusions.

8. The respiratory mask as claimed in claim 1, wherein said second fixing unit includes a second fixing internal ring that is disposed around said first fixing unit, and a second fixing external ring that is formed on said mask body around said second fixing internal ring and that engages said second fixing internal ring, said outer periphery of said damping ring being clamped between said second fixing internal and external rings.

9. The respiratory mask as claimed in claim 8, wherein said second fixing internal ring has an outer surface that is formed with an external screw thread, said second fixing external ring having an inner surface that is formed with an internal screw thread engaging threadedly said external screw thread of said second fixing internal ring.

10. The respiratory mask as claimed in claim 9, wherein said mask body has a face contact portion, and a tube connection portion that is connected to said face contact portion and that is formed with said second fixing external ring, said second fixing external ring having said inner surface thereof defining said connecting hole and further having an annular bearing face that projects from said inner surface thereof into said connecting hole in proximity to said damping ring, said second fixing internal ring further having a second press end that extends axially toward said annular bearing face so as to press said outer periphery of said damping ring against said annular bearing face.

11. The respiratory mask as claimed in claim 10, wherein said second fixing external ring further has a distal end that is opposite to said annular bearing face, said second fixing internal ring further having a second handling end portion that is opposite to said second press end, and that projects outwardly and radially to extend over said distal end, said second handling end portion being operable to rotate said second fixing internal ring.

12. The respiratory mask as claimed in claim 1, wherein said damping ring further has a folded flexible intermediate web that interconnects said inner and outer peripheries.

13. The respiratory mask as claimed in claim 1, wherein said damping ring is made of silicone rubber.
